(19) United States
(12) Patent Application Publication
(10) Pub. No.: US 2025/0345122 A1
(43) Pub. Date: Nov. 13, 2025

(54) SENSOR FUSION METHOD AND APPARATUS CAPABLE OF SIMULTANEOUSLY MEASURING REAL-TIME SHAPE DEFORMATION AND TRACKING TOOL POSE

(71) Applicants: KOREA INSTITUTE OF SCIENCE AND TECHNOLOGY, Seoul (KR); SAMSUNG LIFE PUBLIC WELFARE FOUNDATION, Seoul (KR)

(72) Inventors: Sunghwan LIM, Seoul (KR); Yonggi JUNG, Seoul (KR); Sangseo JEON, Seoul (KR); Thandar Hnin KHAING, Seoul (KR)

(73) Assignees: KOREA INSTITUTE OF SCIENCE AND TECHNOLOGY, Seoul (KR); SAMSUNG LIFE PUBLIC WELFARE FOUNDATION, Seoul (KR)

(21) Appl. No.: 19/200,978

(22) Filed: May 7, 2025

(30) Foreign Application Priority Data
May 8, 2024 (KR) 10-2024-0060318

(51) Int. Cl. A61B 34/20; A61B 90/00

(52) U.S. Cl. CPC A61B 34/20; A61B 90/39; A61B 2034/2055; A61B 2034/2065; A61B 2090/3937; A61B 2560/0223

(57) ABSTRACT

Provided is a method for fusing heterogeneous sensors having a plurality of depth sensors and a three-dimensional localizer and capable of simultaneous real-time human body deformation measurement and medical instrument tracking. The method may include setting a separation position and angle of the plurality of depth sensors and the three-dimensional localizer, performing a one-time calibration to estimate a relationship between coordinate systems of the plurality of depth sensors and the three-dimensional localizer, which are fixed to a frame according to the set separation position and angle, integrating data of the plurality of depth sensors and the three-dimensional localizer into a single coordinate system using the one-time calibration result, and simultaneously measuring real-time surface shape deformation of a target object and tracking positions and orientations of medical instruments using the integrated single coordinate system.

### CROSS-REFERENCE TO RELATED APPLICATION

This application claims the benefit of and priority to Korean Patent Application No. 10-2024-0060318, filed on May 8, 2024, the entire disclosure(s) of which is hereby incorporated herein by reference in its entirety.

### BACKGROUND

### Field

The present disclosure relates to a method and apparatus for fusing heterogeneous sensors and, more particularly, to a heterogeneous sensor fusion method and apparatus capable of simultaneously measuring real-time shape deformation of a target object and the positions and orientations of tools.

### Description of Related Art

Computer-based digital technology is being rapidly and widely adopted in a medical field, and is leading to revolutionary changes in patient treatment. In particular, numerous efforts are being made to utilize digital technology in an operating room, which leads to a research field called Computer-Integrated Surgery (CIS).

Among various areas of the research field of CIS, image-guided surgery, procedural navigation and robotic systems are some of the most prominent technologies. These surgical systems can provide intuitive visual guidance and real-time feedback to a surgeon by utilizing medical images obtained before surgery and real-time sensor data obtained during surgery.

These systems enable the precise manipulation of a surgical/procedural tool by finding the exact position of a predetermined surgical/procedural plan and, ultimately greatly enhance the accuracy and safety of the surgery/procedure.

Image-guided navigation and robotic technologies are widely adopted across various surgical/procedural fields. Areas where this navigation technology is being applied include spinal intervention, spinal fusion surgery, partial or total knee replacement surgery, total hip replacement surgery, femoral fracture reduction surgery, cranio-maxillo-facial surgery, and otolaryngologic surgery.

However, these technologies have common limitations. Most of them have been developed for hard tissues such as bone. Due to these limitations, they cannot be applied to flexible and deformable targets.

Therefore, to solve the problems of the prior art, there is a growing need for a CIS system that can monitor flexible human organs (soft tissues) in real time. In particular, in surgeries involving the breast, face, or incised skin, the system should be able to provide intuitive and accurate guidance to surgeons and practitioners by ensuring accurate position/orientation tracking of surgical/procedural tools while performing real-time shape measurements of the surgical target that is deformed in real time.

### SUMMARY

The present disclosure provides a method and apparatus performing the fusion of heterogeneous sensors.

The present disclosure provides a method and apparatus performing the fusion of heterogeneous sensors, thereby measuring deformation that may occur in a patient's body part in real time, and accurately tracking the positions and orientations of surgical or procedural tools.

In an aspect, a method for fusing heterogeneous sensors having a plurality of depth sensors and a three-dimensional localizer and capable of real-time human body deformation measurement and medical instrument tracking may include setting a separation position and angle of the plurality of depth sensors and the three-dimensional localizer; performing a one-time calibration to estimate a relationship between coordinate systems of the plurality of depth sensors and the three-dimensional localizer, which are fixed to a frame according to the set separation position and angle; integrating data of the plurality of depth sensors and the three-dimensional localizer into a single coordinate system using the one-time calibration result; and simultaneously measuring real-time surface shape deformation of a target object and tracking positions and orientations of tools using the integrated single coordinate system.

The setting the separation position and angle of the plurality of depth sensors and the three-dimensional localizer can set the separation position and angle of the plurality of depth sensors and the three-dimensional localizer to satisfy conditions such as ensuring that the plurality of depth sensors do not block the field of view of the three-dimensional localizer, minimizing the size of the entire sensor system in which the plurality of depth sensors and the three-dimensional localizer are integrated, and minimizing the blind spot of an individual depth sensor among the plurality of depth sensors, where measurement is not possible. At this time, the separation position and angle of the plurality of depth sensors may be set in consideration of the convergence angle between the plurality of depth sensors according to the separation distance between the plurality of depth sensors and a target object sensed by the plurality of depth sensors. Using data on the separation position and angle of the plurality of depth sensors and the three-dimensional localizer set in the above step, a rigid frame for fixing a sensor is designed and manufactured, and the plurality of depth sensors and the three-dimensional localizer may be fixed to the rigid frame.

The performing the one-time calibration may include measuring the surface points of a calibration plate, to which three or more retroreflective markers are attached and fixed at any position, based on each depth sensor coordinate system, using the plurality of depth sensors; calculating a normal vector of the calibration plate based on each depth sensor coordinate system using the surface points measured by each depth sensor; measuring three-dimensional positions of the three or more retroreflective markers based on the three-dimensional localizer coordinate system using the three-dimensional localizer; calculating a normal vector of the calibration plate based on the three-dimensional localizer coordinate system using the three-dimensional positions of the retroreflective markers measured by the three-dimensional localizer; calculating a plurality of three-dimensional rotation matrices using a plurality of normal vector sets obtained by repeatedly performing the step of measuring and receiving the surface shape to the step of calculating the normal vector two or more times; calculating a plurality of three-dimensional translation vectors using the plurality of normal vector sets, the plurality of three-dimensional rotation matrices, the surface points of the calibration plate, and three-dimensional positions of the retroreflective markers; and generating a plurality of rigid body transformation matrices from the three-dimensional localizer coordinate system to the coordinate system of each of the plurality of depth sensors using the three-dimensional rotation matrix and the three-dimensional translation vector obtained through the calculation.

The integrating the data of the plurality of depth sensors and the three-dimensional localizer into the single coordinate system may include measuring surface shape data based on each depth sensor coordinate system from the plurality of depth sensors; and transforming the surface shape data based on the single coordinate system set as the coordinate system of the three-dimensional localizer using the rigid body transformation matrix obtained in the one-time calibration process.

The measuring the real-time surface shape deformation of the target object and tracking positions and orientations of tools may include obtaining surface shape measurement data transformed based on the single coordinate system; and obtaining data on the positions and orientations of the tools represented based on the single coordinate system. The obtaining the surface shape measurement data and the obtaining the data on the positions and orientations of the tools may be simultaneously and repeatedly performed using parallel operation.

The method may further include visualizing data measuring the real-time surface shape deformation of the target object and data simultaneously tracking the positions and orientations of the tools.

The visualizing the data may include rendering three-dimensional models of the tools in a virtual space through three-dimensional positions of a retroreflective markers attached to the tools; rendering a three-dimensional model of the target object in a virtual space through three-dimensional surface shape information; and updating the positions and orientations of the three-dimensional models in real time.

The visualizing the data may further include checking whether the retroreflective marker attached to the tool is visible, thereby visually expressing the dislodgement of the tool when the tool is out of the field of view of a fusion apparatus of the heterogeneous sensors.

The plurality of depth sensors and the three-dimensional localizer can be configured in an orthogonally polarized state based on the similarity between (i) a wavelength range of light used by the plurality of depth sensors and (ii) a wavelength range of light used by the three-dimensional localizer.

In an aspect, an apparatus for fusing heterogeneous sensors may include a plurality of depth sensors; a three-dimensional localizer; and a processor. The processor may perform a one-time calibration to estimate a relationship between coordinate systems of the plurality of depth sensors and the three-dimensional localizer, which are fixed to the frame, integrate data of the plurality of depth sensors and the three-dimensional localizer into a single coordinate system using the one-time calibration result, and simultaneously measure real-time surface shape deformation of a target object, and tracks positions and orientations of tools.

The separation position and angle may be set, in consideration of a convergence angle between the plurality of depth sensors according to a separation distance between the plurality of depth sensors and a target object sensed by the plurality of depth sensors.

The processor may measure the surface points of a calibration plate, to which three or more retroreflective markers are attached and fixed at any position, based on each depth sensor coordinate system, using the plurality of depth sensors, calculate a normal vector of the calibration plate based on each depth sensor coordinate system using the surface points measured by each depth sensor, measure three-dimensional positions of the three or more retroreflective markers based on the three-dimensional localizer coordinate system using the three-dimensional localizer, calculate a normal vector of the calibration plate based on the three-dimensional localizer coordinate system using the three-dimensional positions of the retroreflective markers measured by the three-dimensional localizer, calculate a plurality of three-dimensional rotation matrices using a plurality of normal vector sets obtained by repeating the process of calculating the normal vector, calculate a plurality of three-dimensional translation vectors using i) the plurality of normal vector sets, ii) the plurality of three-dimensional rotation matrices, iii) the surface points of the calibration plate, and iv) three-dimensional positions of the retroreflective markers, and generate a plurality of rigid body transformation matrices from the three-dimensional localizer coordinate system to the coordinate system of each of the plurality of depth sensors using the three-dimensional rotation matrix and the three-dimensional translation vector obtained through the calculation.

The processor may measure surface shape data based on each depth sensor coordinate system from the plurality of depth sensors, and transform the input surface shape data based on the single coordinate system set as the coordinate system of the three-dimensional localizer using the rigid body transformation matrix obtained in the one-time calibration process.

The processor may obtain surface shape measurement data transformed based on the single coordinate system, and obtain data on the positions and orientations of the tools represented based on the single coordinate system, thereby measuring surface shape deformation in real time and tracking the positions and orientations of the tools.

The processor may simultaneously and repeatedly perform, by the heterogeneous sensor fusion apparatus, a process of obtaining the surface shape measurement data and a process of obtaining the data on the positions and orientations of the tools through parallel operation.

The processor may visualize data simultaneously measuring the real-time surface shape deformation of the target object and tracking the positions and orientations of the tools.

The processor may render three-dimensional models of the tools in a virtual space through the three-dimensional positions of the retroreflective markers attached to the tools, and update the positions and orientations of the three-dimensional models of the tools in real time.

The processor may check whether the retroreflective marker attached to the tool is visible, thereby visually expressing the dislodgement of the tool when the tool is out of the field of view of the fusion apparatus of the heterogeneous sensors.

The present disclosure can provide a heterogeneous sensor fusion method and apparatus, capable of simultaneously measuring real-time surface shape deformation and the positions and orientations of tools.

The present disclosure can provide a heterogeneous sensor fusion method and apparatus, capable of simultaneously measuring real-time surface shape deformation and the positions and orientations of tools, thereby allowing surface shape deformation that may occur in a patient's body part and the positions and orientations of medical instruments used in a surgery or procedure to be accurately and simultaneously measured in real time.

The present disclosure can provide a heterogeneous sensor fusion method and apparatus, capable of simultaneously measuring real-time surface shape deformation and the positions and orientations of tools, so that it can be utilized in an image-guided surgery or procedure targeting a patient's deformed body part, a surgical or procedure navigation and robot, or a surgical or procedural automation system.

The plurality of depth sensors and the three-dimensional localizer can be configured in an orthogonally polarized state based on the similarity between (i) a wavelength range of light used by the plurality of depth sensors and (ii) a wavelength range of light used by the three-dimensional localizer. At this time, the orthogonally polarized state is based on polarizing films or polarizing lenses attached to an illumination part or a camera unit of each of the plurality of depth sensors and the three-dimensional localizer.

### BRIEF DESCRIPTION OF THE DRAWING

FIG. 1 is a flowchart illustrating a method for fusing heterogeneous sensors according to an embodiment of the present disclosure.

FIG. 2 is a flowchart illustrating a one-time calibration method for estimating a relationship between heterogeneous sensor coordinate systems.

FIG. 3 is a diagram illustrating a method of setting the separation positions and angles of a plurality of depth sensors in heterogeneous sensors.

FIG. 4 is a diagram illustrating a one-time calibration method of heterogeneous sensors.

FIG. 5 is a diagram illustrating a method of managing and visualizing data acquired through the heterogeneous sensors.

FIG. 6 is a diagram illustrating an apparatus for fusing heterogeneous sensors according to an embodiment of the present disclosure.

### DETAILED DESCRIPTION

Hereinafter, embodiments of the present disclosure will be described in detail with reference to the accompanying drawings so that a person having ordinary skill in the art to which the present disclosure pertains can easily implement the present disclosure. However, the present disclosure may be implemented in many different forms and is not limited to the embodiments described herein.

In describing embodiments of the present disclosure, if it is determined that a specific description of a known configuration or function may obscure the gist of the present disclosure, a detailed description thereof will be omitted. In addition, parts that are not related to the description of the present disclosure will be omitted in the drawings, and like reference numerals refer to like parts throughout various figures and embodiments of the present disclosure.

It will be understood that when a component is referred to as being “coupled” or “connected” to another component, it can be directly coupled or connected to the other component or intervening components may be present therebetween. Further, when a certain component “includes” or “has” another component, it means, unless otherwise explicitly stated, that it does not exclude other components but may include additional components as well.

It will be understood that, although the terms “first”, “second”, etc. may be used herein to describe various components, these components should not be limited by these terms. These terms are only used to distinguish one component from another component. For instance, a first component discussed below could be termed a second component without departing from the teachings of the present disclosure. Similarly, the second component could also be termed the first component.

In this disclosure, distinct components are used to clearly explain each characteristic, and do not necessarily mean that the components are separated. That is, multiple components may be integrated to form a single hardware or software unit, or a single component may be distributed to form multiple hardware or software units. Thus, unless otherwise stated, such integrated or distributed embodiments are also included in the scope of the present disclosure.

In the present disclosure, components described in various embodiments do not necessarily mean essential components, and some may be optional components. Thus, embodiments comprising a subset of components described in one embodiment are also included within the scope of the present disclosure. Further, embodiments including other components as well as components described in the various embodiments are also included within the scope of the present disclosure.

The above and other objectives, features, and advantages of the present disclosure will be easily understood from the following preferred embodiments in conjunction with the accompanying drawings. However, the disclosure may be embodied in different forms without being limited to the embodiments set forth herein. Rather, the embodiments disclosed herein are provided to make the disclosure thorough and complete and to sufficiently convey the spirit of the present disclosure to those skilled in the art.

The terminology used herein is for the purpose of describing particular embodiments only and is not intended to be limiting. In the present disclosure, the singular forms are intended to include the plural forms as well, unless the context clearly indicates otherwise. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

Unless otherwise defined, all terms including technical and scientific terms used herein have the same meaning as commonly understood by one of ordinary skill in the art to which the present disclosure belongs. It will be further understood that terms used herein should be interpreted as having a meaning that is consistent with their meaning in the context of this specification and the relevant art and will not be interpreted in an idealized or overly formal sense unless expressly so defined herein.

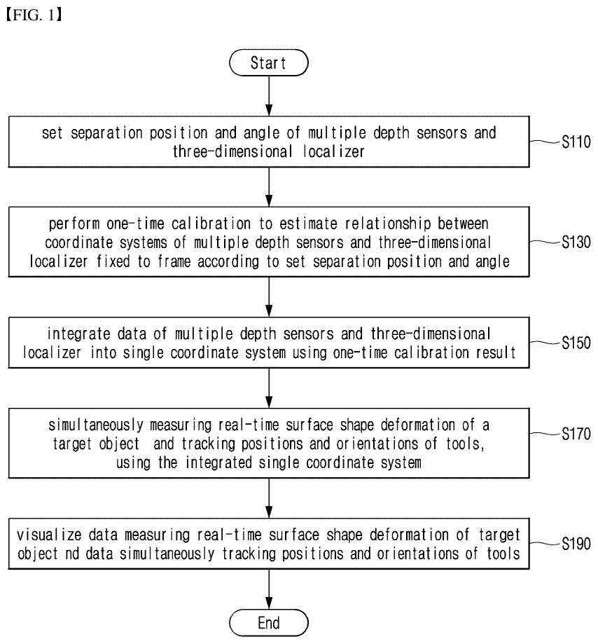

FIG. 1 is a flowchart illustrating a method for fusing heterogeneous sensors according to an embodiment of the present disclosure.

Referring to FIG. 1, in step S110, a heterogeneous sensor fusion apparatus including a plurality of depth sensors and a three-dimensional localizer may set the separation positions and angles of the plurality of depth sensors and the three-dimensional localizer.

In step S130, the heterogeneous sensor fusion apparatus may perform one-time calibration to estimate a relationship between coordinate systems of the plurality of depth sensors fixed to a frame and the three-dimensional localizer according to the set separation positions and angles.

In step S150, the heterogeneous sensor fusion apparatus may integrate data from the plurality of depth sensors and the three-dimensional localizer into a single coordinate system using the one-time calibration result.

In the process of integrating the data from the plurality of depth sensors and the three-dimensional localizer into the single coordinate system, the heterogeneous sensor fusion apparatus may receive surface shape data based on each depth sensor coordinate system measured from the plurality of depth sensors, and transform the input surface shape data based on the single coordinate system that is set as the coordinate system of the three-dimensional localizer using a rigid body transformation matrix obtained in the one-time calibration process.

In step S170, the heterogeneous sensor fusion apparatus may simultaneously measure the real-time surface shape deformation of a target object and tracking positions and orientations of tools, using the integrated single coordinate system.

In the process of simultaneously measuring the real-time surface shape deformation of the target object and tracking the positions and orientations of the tools, the heterogeneous sensor fusion apparatus may obtain surface shape measurement data converted based on the single coordinate system, and obtain data on the positions and orientations of the tools expressed based on the single coordinate system. At this time, the acquisition of the surface shape measurement data and the acquisition of the position and orientations data of the tools may be performed simultaneously and repeatedly using parallel operation.

In step S190, the heterogeneous sensor fusion apparatus according to an embodiment of the present disclosure may visualize data measuring the real-time surface shape deformation of the target object and data simultaneously tracking the positions and orientations of the tools.

A detailed description of each step and a description of the heterogeneous sensor fusion apparatus will be supplemented by the description of the drawings below.

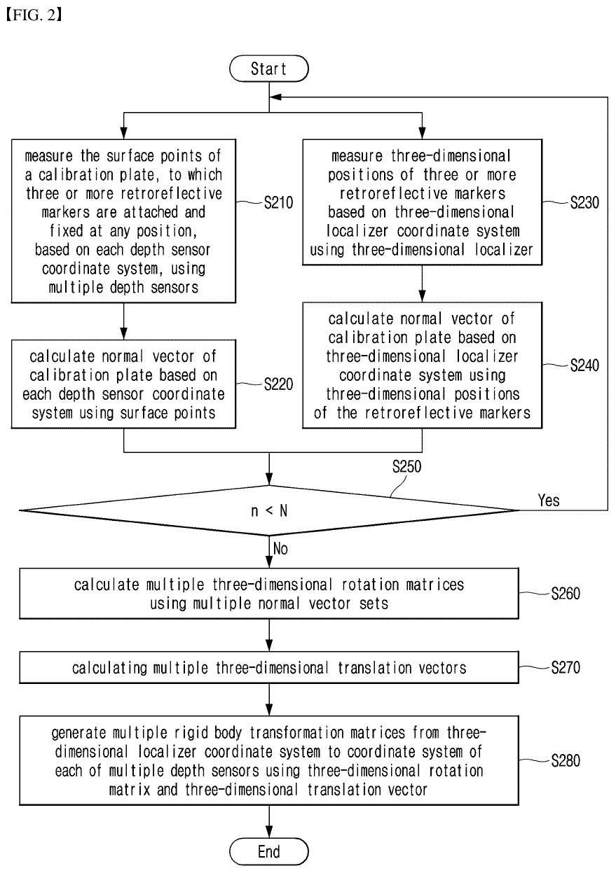

FIG. 2 is a flowchart illustrating a one-time calibration method for estimating a relationship between heterogeneous sensor coordinate systems.

Referring to FIG. 2, in step S210, the heterogeneous sensor fusion apparatus may measure the surface points of a calibration plate, to which three or more retroreflective markers are attached and fixed at any position, based on each depth sensor coordinate system, using a plurality of depth sensors.

In step S220, the heterogeneous sensor fusion apparatus may calculate the normal vector of the calibration plate based on each depth sensor coordinate system using the surface points measured by each depth sensor.

In step S230, the heterogeneous sensor fusion apparatus may measure the three-dimensional positions of the three or more retroreflective markers based on a three-dimensional localizer coordinate system, using the three-dimensional localizer.

In step S240, the heterogeneous sensor fusion apparatus may calculate a normal vector of the calibration plate based on the three-dimensional localizer coordinate system, using the three-dimensional position.

In step S250, the heterogeneous sensor fusion apparatus may repeatedly perform the step of calculating the normal vector two or more times. Here, n represents an index for the number of movements of the calibration plate, and n is set to 1, when the calibration plate is first fixed at any position and orientation. As the step of calculating the normal vector is repeated, the calibration plate is newly fixed at any position and orientations, and n increases by 1 (n=n+1). N represents the total number of movements of the calibration plate or the total number of data measurements using each sensor in the heterogeneous sensors (N is a natural number greater than or equal to 2).

In step S260, the heterogeneous sensor fusion apparatus may calculate a plurality of three-dimensional rotation matrices using a plurality of normal vector sets obtained through the above repetition.

In step S270, the heterogeneous sensor fusion apparatus may calculate a plurality of three-dimensional translation vectors using i) the plurality of normal vector sets ii) the plurality of three-dimensional rotation matrices, iii) the surface points of the calibration plate, and iv) three-dimensional positions of the retroreflective markers.

In step S280, the heterogeneous sensor fusion apparatus may generate a plurality of rigid body transformation matrices from the three-dimensional localizer coordinate system to the coordinate system of each of the plurality of depth sensors using the three-dimensional rotation matrix and the three-dimensional translation vector obtained through the calculation.

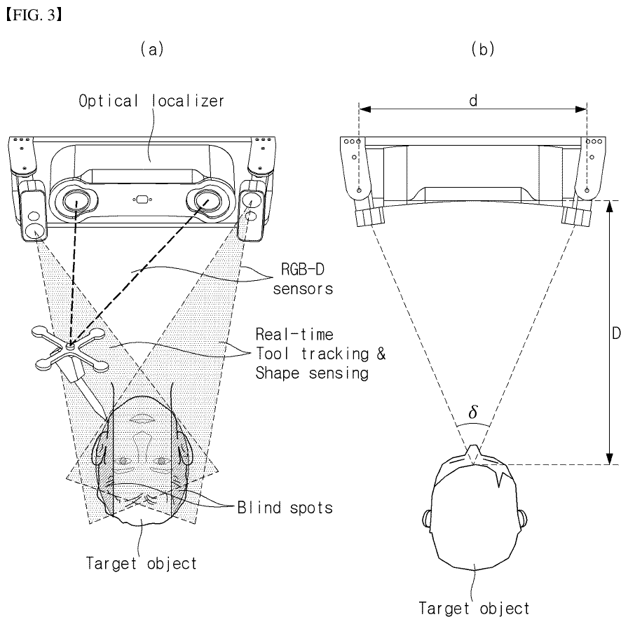

FIG. 3 is a diagram illustrating a method of setting the separation positions and angles of the plurality of depth sensors in heterogeneous sensors.

Referring to FIG. 3, the heterogeneous sensor fusion apparatus may adjust the separation positions and angles of the plurality of depth sensors in the heterogeneous sensors. At this time, each component sensor of the heterogeneous sensor may preferably include a plurality of depth sensors and a three-dimensional localizer. The plurality of depth sensors may include an RGB-D sensor, and the three-dimensional localizer may include an optical localizer.

Referring to FIG. 3(a), the optical localizer in the heterogeneous sensors may detect and track the six degrees of freedom (6DoF) pose of a medical device or medical tool, which are the six movements of an object combining rotational motion in three directions and translational motion moving parallel in a three-dimensional space). The optical localizer may calculate the 6DoF pose of the medical device or medical tool by emitting infrared light and detecting the infrared light reflected from the retroreflective markers attached to the medical device or medical tool.

A plurality of RGB-D sensors attached to the right and left sides of the optical localizer in the heterogeneous sensors may capture images of a patient's body parts using a visible-light camera and a Time of Flight (ToF) sensor to measure three-dimensional surface data and corresponding RGB color information of body parts that may be deformed during surgery or procedures.

The plurality of depth sensors and the three-dimensional localizer may be configured in an orthogonally polarized state based on the similarity between (i) a wavelength range of light used by the plurality of depth sensors and (ii) a wavelength range of light used by the three-dimensional localizer.

For example, the plurality of depth sensors and the three-dimensional localizer may be configured such that i) the light used by the plurality of depth sensors and ii) the light used by the three-dimensional localizer are in an orthogonally polarized state.

When the optical localizer and RGB-D sensors in the heterogeneous sensor utilize light in a similar wavelength range, such as near-infrared, an orthogonally polarized state can be achieved to prevent sensor interference. For example, the orthogonally polarized state may be achieved by attaching polarizing film or polarizing lenses to the illumination or camera unit of each sensor.

Referring to FIG. 3(b), since there are multiple RGB-D sensors in the heterogeneous sensors, it can help expand the field of view when measuring a patient's body part, as well as minimize blind spots that occur when relying on a single RGB-D sensor. The blind spots where data may not be measured by the single RGB-D sensor may include a case where the field of view is obstructed by an operator's hand or a surgical tool, or a case where measurements may not be made with the field of view of the single RGB-D sensor due to the curved shape of a body part.

A convergence angle δ between the plurality of RGB-D sensors in the heterogeneous sensors may be determined by a distance D from the heterogeneous sensor to a sensing target object and a separation distance d between the plurality of RGB-D sensors.

Considering a surgical environment in an operating room, it may be preferred to maximize the distance D from the heterogeneous sensors to the sensing target object. To this end, by placing the heterogeneous sensors further away from a surgical target, medical personnel may secure a greater range of motion, creating a more comfortable surgical working environment. Such a working environment has the advantage of reducing the risk of bacterial infection for patients. However, considering the accuracy of the RGB-D sensor and the optical localizer in the heterogeneous sensors, the closer the distance D from the heterogeneous sensors to the sensing target object, the more advantageous it may be to reduce potential measurement errors of the heterogeneous sensors. That is, the accuracy of the plurality of RGB-D sensors and the distance D from the heterogeneous sensors to the sensing target object are inversely proportional to each other. Considering this relationship, the distance D to the sensing target object may be set.

Further, the separation distance d between the plurality of RGB-D sensors in the heterogeneous sensors may be advantageously increased so that the plurality of RGB-D sensors in the heterogeneous sensors do not obstruct the field of view of the optical localizer and the blind spot of the single RGB-D sensor is minimized. However, the smaller the size of the heterogeneous sensor fusion apparatus, the easier it may be to use in an operating room environment. Thus, the separation distance (between the plurality of RGB-D sensors may be set.

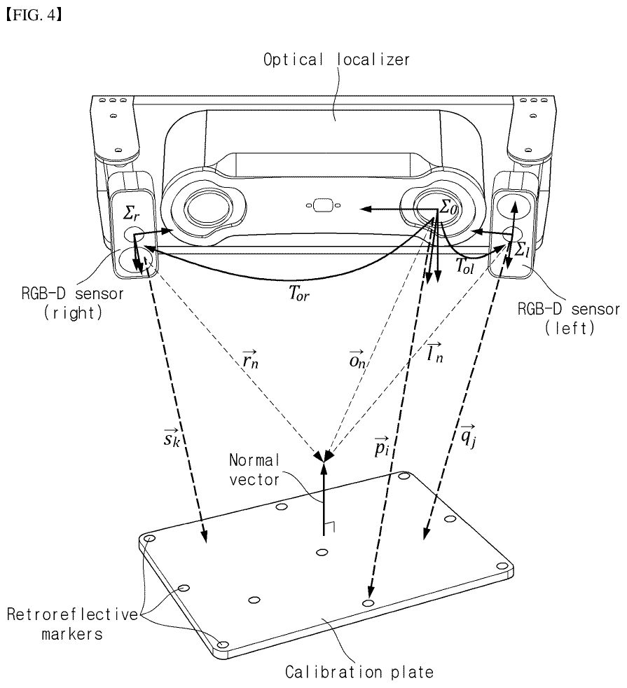

FIG. 4 is a diagram illustrating a one-time calibration method of the heterogeneous sensors.

Referring to FIG. 4, the one-time calibration may be performed to estimate each three-dimensional rigid transformation matrix between a plurality of RGB-D sensor coordinate systems (Σₗ, Σᵣ) fixed to the rigid frame and an optical localizer coordinate system (Σₒ). To this end, a single color calibration plate to which three or more retroreflective markers are attached may be used. The plurality of RGB-D sensors may estimate one or more normal vectors by measuring the planar shape of the calibration plate fixed at any position and orientation. The optical localizer may measure one or more normal vectors by measuring the positions of three or more retroreflective markers on the calibration plate in real time. The above normal vector estimation may be repeatedly performed at least twice while changing the position and orientation of the calibration plate. Using the normal vector set obtained in this way, the three-dimensional rigid body transformation matrix (Tₒₗ, Tₒᵣ) bet between respective sensor coordinate systems may be estimated.

The calibration plate may be painted in a single color that is easily distinguishable from the surrounding environment, and the surface of the calibration plate may have three or more retroreflective markers attached at any positions.

The calibration plate may be fixed at any position and orientation, surface shape data (\vec{q}ⱼ, \vec{s}ₖ) of the calibration plate may be measured with the plurality of RGB-D sensors, and three-dimensional positions (\vec{p}ᵢ) of the three or more retroreflective markers attached to the calibration plate may be measured with the optical localizer. At this time, i, j, k represent the index of the surface shape data measured from the first RGB-D sensor, the surface shape data measured from the second RGB-D sensor, and the position data number of the retroreflective marker measured from the optical localizer, respectively. Using the measured data, each normal vector \vec{O}ₙ, \vec{l}ₙ, \vec{r}ₙ defined in each sensor coordinate system may be estimated.

At this time, the calibration plate is fixed at another position and orientation, and data measurement is repeated at least once, so that each normal vector \vec{O}ₙ, \vec{l}ₙ, \vec{r}ₙ defined in each sensor coordinate system may be repeatedly estimated. At this time, n represents an index for the number of movements of the calibration plate.

Using the center point data \vec{p}ᵢ ([xᵢ, yᵢ, xᵢ]^{T}) of the retroreflective marker measured by the optical localizer in the heterogeneous sensors and the plane equation (ax+by+C=z), the normal vector (\vec{O}ₙ) of the calibration plate in the optical localizer coordinate system may be estimated as shown in Equation 1 below.

$$[ \begin{matrix}⋮ & ⋮ & ⋮ \\ {x}_{i} & {y}_{i} & 1 \\ ⋮ & ⋮ & ⋮\end{matrix} ] [ \begin{matrix}a \\ b \\ c\end{matrix} ] = [ \begin{matrix}⋮ \\ {z}_{i} \\ ⋮\end{matrix} ] \qquad [ Equation 1 ]$$

Referring to Equation 1, the normal vector (\vec{O}ₙ) estimated by the optical localizer is calculated as \vec{x}/∥\vec{x}∥, and means [a, b, c]^{T}. The normal vectors (\vec{l}ₙ, \vec{e}ₙ) the calibration plate in the plurality of RGB-D sensor coordinate systems may also be estimated using the same method through Equation 1.

The heterogeneous sensor fusion apparatus may calculate one or more rotation matrices using the vector set (\vec{O}ₙ, \vec{l}ₙ, \vec{r}ₙ) obtained in the above process, calculate one or more translation vectors using the matrices, and obtain a three-dimensional rigid body transformation matrix between the sensor coordinate systems based on one or more rotation matrices and one or more translation vectors.

The heterogeneous sensor fusion apparatus may calculate the rotation transformation matrix (Rₒₗ, Rₒᵣ) between the sensor coordinate systems in the heterogeneous sensors using the estimation normal vector (\vec{O}ₙ, \vec{l}ₙ, \vec{r}ₙ).

The heterogeneous sensor fusion apparatus may calculate a matrix H(H) using the normal vector (\vec{O}ₙ, \vec{l}ₙ)) as in Equation 2. Referring to Equation 2, N represents the total number of movements of the calibration plate or the total number of data measurements using each sensor in the heterogeneous sensors.

$$H = {\sum }_{n = 1}^{N} {\vec{l}}_{n} {\vec{o}}_{n}^{T} \qquad [ Equation 2 ]$$

Referring to Equation 3, the heterogeneous sensor fusion apparatus may calculate the rotation transformation matrix Rₒₗ between the optical localizer coordinate system and a first RGB-D sensor coordinate system by applying singular value decomposition to the matrix H(H) to decompose the matrix H (H) into UΣV^{T}. The heterogeneous sensor fusion apparatus may calculate the rotation transformation matrix between the optical localizer coordinate system and a second RGB-D sensor coordinate system in the same method as the method of calculating the rotation transformation matrix Rₒᵣ using the normal vector \vec{O}ₙ, \vec{r}ₙ. The above process may be repeated for the plurality of RGB-D sensors.

$${R}_{ol} = V [ \begin{matrix}1 & 0 & 0 \\ 0 & 1 & 0 \\ 0 & 0 & det ( {VU}^{T} )\end{matrix} ] {U}^{T} \qquad [ Equation 3 ]$$

That is, the heterogeneous sensor fusion apparatus may measure data at least twice using one or more depth sensors, the optical localizer, and the calibration plate, calculate one or more matrices H using a set of normal vectors estimated through the measurement, and calculate one or more rotation transformation matrices by applying the singular value decomposition to each of the one or more matrices H.

The heterogeneous sensor fusion apparatus may use a constraint that ensures the vector between the normal vector \vec{q}′ⱼ(\vec{q}′ⱼ=Rₒₗ\vec{q}ⱼ+tₒₗ), obtained by transforming the surface shape data \vec{q}ⱼ of the calibration plate measured by the first RGB-D sensor using the rotation transformation matrix Rₒₗ, and the retroreflective marker position data \vec{p}ᵢ measured by the optical localizer in the heterogeneous sensors, is perpendicular to the estimated normal vector \vec{O}ₙ measured by the optical localizer, to estimate the translation vector \vec{t}ₒₗ between the coordinate system of the optical localizer and the coordinate system of the first RGB-D sensor. The characteristics of the vertical constraint are as shown in Equation 4 below.

$${\vec{o}}_{n}^{T} ( {\vec{p}}_{i} - {{\vec{q}}^{′}}_{j} ) = 0 \qquad [ Equation 4 ]$$

If \vec{q}′ⱼ=Rₒₗ\vec{q}ⱼ+\vec{t}ₒₗ is substituted into Equation 4, it is the same as Equation 5 below.

$${\vec{o}}_{n}^{T} {\vec{t}}_{ol} = {\vec{o}}_{n}^{T} ( {\vec{p}}_{i} - {R}_{ol} {\vec{q}}_{j} ) \qquad [ Equation 5 ]$$

Based on Equation 5, when Equation 5 is accumulated for i=1, . . . , 1 j=1, . . . , J n=1, . . . , N 1 the heterogeneous sensor fusion apparatus may obtain the least square equation, which is Equation 6 below. At this time, I means the number of retroreflective markers measured using the optical localizer, J means the number of calibration plate surface shape data measured using the first RGB-D sensor, and N means a total number of movements of the calibration plate or a total number of data measurements using each sensor in the heterogeneous sensors.

$$A {\vec{t}}_{ol} = \vec{b} \qquad [ Equation 6 ]$$

Referring to Equation 6, A has the form of [symbol] ^{IJN×3}, and \vec{b} has the form of [symbol] ^{IJN} Thereby, the heterogeneous sensor fusion apparatus may estimate the translation vector \vec{t}^{ol}, and estimate the translation vector \vec{t}^{or} using Equations 4 to 6 in the same way.

The heterogeneous sensor fusion apparatus may generate the rigid transformation matrix Tₒₗ([Rₒₗ, \vec{t}ₒₗ]) from the optical localizer coordinate system to the first RGB-D sensor coordinate system, using the rotation matrix Rₒₗ acquired using the first RGB-D sensor and the optical localizer, and the translation vector \vec{t}ₒₗ.

The heterogeneous sensor fusion apparatus may generate the rigid transformation matrix Tₒᵣ(Rₒᵣ, \vec{t}ₒᵣ) from the optical localizer coordinate system to the second RGB-D sensor coordinate system, using the rotation matrix Rₒᵣ acquired using the second RGB-D sensor and the optical localizer, and the translation vector \vec{t}ₒᵣ. The above operation may be repeatedly performed on the plurality of RGB-D sensors.

That is, the heterogeneous sensor fusion apparatus may generate one or more transformation normal vectors by transforming one or more normal vectors using one or more rotation matrices, calculate one or more translation vectors based on one or more normal vectors and one or more transformation normal vectors, and generate one or more rigid body transformation matrices based on one or more rotation matrices and one or more translation vectors.

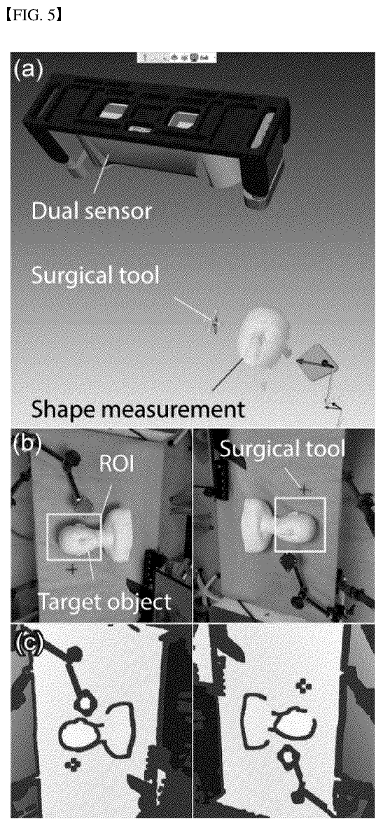

FIG. 5 is a diagram illustrating a method of managing and visualizing data acquired through the heterogeneous sensors.

Referring to FIG. 5, in order to manage and visualize sensor data acquired through the plurality of depth sensors and the three-dimensional localizer in the heterogeneous sensors, the heterogeneous sensor fusion apparatus may have a separate software program within the heterogeneous sensor fusion apparatus, or a processor may visualize data obtained through the heterogeneous sensors, in the heterogeneous sensor fusion apparatus.

The software program may perform down-sampling, edge noise reduction, region of interest (Rₒₗ) localization, target segmentation, point cloud fusion, and tool tracking based on the sensor data.

The software program may uniformly down-sample the raw depth data measured via the heterogeneous sensors, taking into account that different regions of the human body do not have complex geometries. Through the down-sampling, the software program may generate point cloud data sampled at a user-defined ratio, such as 1/4, 1/9, 1/16, or 1/25.

The software program may perform edge noise reduction on depth data measured via the heterogeneous sensors. The depth data measured via the heterogeneous sensors usually includes dynamic noises in an edge. The dynamic noises may also be generated during the post-processing of the heterogeneous sensors in the heterogeneous sensors. Since the depth data does not represent the actual measurement value of a patient's body part, the data is inevitably less reliable.

Therefore, referring to FIG. 5(c), the software program may filter an unreliable measurement value obtained from the edge of an object through a filtering algorithm, to increase the reliability of the depth data. The filtering algorithm may detect the edge within a depth image using a canny edge detection algorithm, and perform a user-specified number of dilation operations on the detected edge in the software program, thereby generating masking data. The software program may generate depth data with the edge removed by masking a depth image within the input depth data using masking data generated through the filtering algorithm.

Referring to FIG. 5(b), the software program may set a Rol in an RGB image acquired via the first RGB-D sensor of the heterogeneous sensors by interacting with a user via an interface (e.g., a mouse) linked to the software program.

The software program may reduce the size of operational data by selectively calculating the Rol of the depth data. Further, the software program may continuously track the Rol of the depth data using a tracking algorithm to compensate for the movement of a target object acquired through the heterogeneous sensors during the procedure. The software program may be implemented using various object tracking algorithms to adapt to different imaging conditions and surgical environments.

The software program may segment the Rol of the patient's body part from the point cloud using an extraction algorithm based on the RGB information acquired via the RGB-D sensor. The extraction algorithm may be initialized with requisite segmentation parameters that may be adjusted in real time. At this time, the requisite segmentation parameters may include a variable T_{seg} that is a positive scalar value meaning the allowed intensity range of the color channel of the RGB information, and a variable ρ_{seg} meaning the allowed intensity ratio of two adjacent color channels in the RGB information. The software program may obtain an intensity value C_{seg}=(r_{seg}, g_{seg}, b_{seg}), of a RGB color channel assigned to a patient's body part in an RGB image by interacting with the user through the interface (e.g., the mouse) linked to the software program. Subsequently, the software program may obtain an assigned RGB color value Cₘ=(rₘ, gₘ, bₘ) satisfying Equations 7 and 8 below through a segmentation process.

$$\vert {r}_{m} - {r}_{seg} \vert , \vert {g}_{m} - {g}_{seg} \vert , \vert {b}_{m} - {b}_{seg} \vert \leq {τ}_{seg} \qquad [ Equation 7 ]$$

$$\frac{\vert \frac{{r}_{m}}{{g}_{m}} - \frac{{r}_{seg}}{{g}_{seg}} \vert}{\frac{{r}_{seg}}{{g}_{seg}}} , \frac{\vert \frac{{g}_{m}}{{b}_{m}} - \frac{{g}_{seg}}{{b}_{seg}} \vert}{\frac{{g}_{seg}}{{b}_{seg}}} , \frac{\vert \frac{{b}_{m}}{{r}_{m}} - \frac{{b}_{seg}}{{r}_{seg}} \vert}{\frac{{b}_{seg}}{{r}_{seg}}} \leq {ρ}_{seg} \qquad [ Equation 8 ]$$

Referring to Equations 7 and 8, m denotes an index of three-dimensional surface data in the Rol of the patient's body part.

The software program may apply a voxel grid filter to the entire point cloud data to remove the overlapping area of point cloud data acquired via the plurality of RGB-D sensors. The software program may generate a three-dimensional voxel grid over the entire point cloud data with a preset three-dimensional voxel size. At this time, all point data contained in each voxel may be replaced with the center point of each 3D voxel grid.

Referring to FIG. 5(a), the software program may render three-dimensional models of medical instruments in a virtual space by utilizing the three-dimensional positions of the retroreflective markers attached to the medical instruments transmitted from the heterogeneous sensor fusion apparatus. At this time, the positions and orientations of the three-dimensional models of the medical instruments may be updated in real time. Further, when the software program checks whether the retroreflective marker is visible and notifies the medical tool when it is out of the field of view of the fusion apparatus of the heterogeneous sensors, the color of the three-dimensional model may be dynamically changed to red to indicate to a doctor or user that the medical tool has been dislodged.

The heterogeneous sensor fusion apparatus of the present disclosure linked with the software program may establish a surgery or procedure plan based on a patient's medical image (e.g., CT, MRI, etc.) that may be obtained before surgery or procedure, and may create a three-dimensional deformable model of a body part to be subjected to surgery or procedure.

The heterogeneous sensor fusion apparatus may perform deformable registration using three-dimensional shape data acquired in real time during surgery or procedure from the RGB-D sensor of the heterogeneous sensors and the three-dimensional deformation model prepared in advance.

The heterogeneous sensor fusion apparatus may predict the positional deformation of a patient's body part, which is a surgical or procedure target, located inside the three-dimensional deformation model as the result of the deformable registration. Further, the heterogeneous sensor fusion apparatus can precisely track real-time position and orientation information of surgical or procedural tools using the optical localizer of the heterogeneous sensors.

Since the heterogeneous sensor fusion apparatus based on the multiple RGB-D sensors and the optical localizer in the heterogeneous sensors shares the same three-dimensional coordinate system, it is possible to track the positions and orientations of the surgical or procedural tools in real time, based on the patient's body part to be subjected to surgery or procedure.

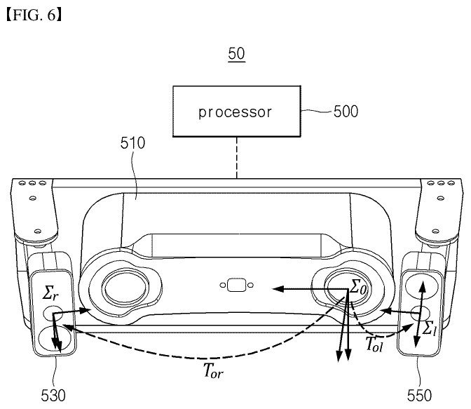

FIG. 6 is a diagram illustrating the heterogeneous sensor fusion apparatus according to an embodiment of the present disclosure. FIG. 6 illustrates the reconstruction of the heterogeneous sensor fusion method in FIG. 1 according to an embodiment of the present disclosure, in terms of hardware configuration. To avoid redundancy in the explanation, only an overview of the operation and function of each component will be described in brief.

Referring to FIG. 6, the heterogeneous sensor fusion apparatus 50 may include a processor 500, a three-dimensional localizer 510, and a plurality of depth sensors 530 and 550.

The plurality of depth sensors 530 and 550 and the three-dimensional localizer 510 may be fixed to the frame according to the set separation position and angle.

The processor 500 may perform one-time calibration to estimate a relationship between the coordinate systems of the plurality of depth sensors 530 and 550 and the three-dimensional localizer 510 fixed to the frame.

The processor 500 may integrate data from the plurality of depth sensors 530 and 550 and the three-dimensional localizer 510 into a single coordinate system using the one-time calibration results.

The processor 500 may measure the deformation of the surface shape in real time using the integrated single coordinate system and simultaneously track the positions and orientations of the tools.

The separation position and the angle may be set to satisfy conditions such as ensuring that the plurality of depth sensors 530 and 550 do not block the field of view of the three-dimensional localizer 510, minimizing the size of the entire sensor system in which the plurality of depth sensors 530 and 550 and the three-dimensional localizer 510 are integrated, and minimizing the blind spot of an individual depth sensor among the plurality of depth sensors 530 and 550, where measurement is not possible. At this time, the separation position and the angle may be set in consideration of the convergence angle between the plurality of depth sensors 530 and 550 according to the separation distance between the plurality of depth sensors 530 and 550 and a target object sensed by the plurality of depth sensors.

The processor 500 may measure and receive the surface shape of the calibration plate to which three or more retroreflective markers are attached and fixed at any position, based on each depth sensor coordinate system using the plurality of depth sensors 530 and 550.

The processor 500 may use each of the input surface shapes to calculate the normal vector of the calibration plate based on each of the depth sensor coordinate systems.

The processor 500 may measure and receive the three-dimensional positions of the three or more retroreflective markers based on the three-dimensional localizer coordinate system using the three-dimensional localizer 510.

The processor 500 may use the input three-dimensional position to calculate the normal vector of the calibration plate based on the three-dimensional localizer coordinate system.

The processor 500 may calculate a plurality of three-dimensional rotation matrices using a plurality of normal vector sets obtained by repeating the process of calculating the normal vectors.

The processor 500 may calculate a plurality of three-dimensional translation vectors using the plurality of normal vector sets and the plurality of three-dimensional rotation matrices.

The processor 500 may generate a plurality of rigid body transformation matrices from the three-dimensional localizer coordinate system to the coordinate system of each of the plurality of depth sensors using the three-dimensional rotation matrix and the three-dimensional translation vector obtained through the operation.

The processor 500 may measure and receive surface shape data based on each depth sensor coordinate system from the plurality of depth sensors 530 and 550, and may transform the input surface shape data based on a single coordinate system, which is set as the coordinate system of the three-dimensional localizer 510, using the rigid body transformation matrix obtained in the one-time calibration process.

The processor 500 may obtain the surface shape measurement data transformed based on the single coordinate system, obtain the position and orientation data of tools expressed based on the single coordinate system, measure the surface shape deformation in real time, and track the position and pose of the tool.

The processor 500 may simultaneously and repeatedly perform the process of obtaining the surface shape measurement data by the heterogeneous sensor fusion apparatus and the process of obtaining the position and orientation data of the tools in parallel operation.

The processor 500 may visualize data measuring the surface shape deformation of the target object in real time and data simultaneously tracking the positions and orientations of the tools.

The processor 500 may render the three-dimensional models of the tools in a virtual space using the three-dimensional positions of the retroreflective markers attached to the tools, and update the positions and orientations of the three-dimensional models of the tools in real time.

When the processor 500 checks whether the retroreflective marker attached to the tool is visible, so the tool is out of the field of view of the fusion apparatus of the heterogeneous sensors, the dislodgement of the tool may be visually expressed.

The plurality of depth sensors 530 and 550 and the three-dimensional localizer 510 may be configured in an orthogonally polarized state based on the similarity between (i) a wavelength range of light used by the plurality of depth sensors 530 and 550 and (ii) a wavelength range of light used by the three-dimensional localizer 510.

For example, the plurality of depth sensors 530 and 550 and the three-dimensional localizer 510 may be configured such that i) the light used by the plurality of depth sensors 530 and 550 and ii) the light used by the three-dimensional localizer 510 are in the orthogonally polarized state.

For example, the orthogonally polarized state is based on polarizing films or polarizing lenses attached to an illumination part or a camera unit of each of the plurality of depth sensors 530 and 550 and the three-dimensional localizer 510.

Embodiments of the present disclosure may be implemented by various means, for example, hardware, firmware, software, or a combination thereof. For hardware implementation, an embodiment of the present disclosure may be implemented by one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, and the like. For implementation in firmware or software, an embodiment of the present disclosure may be implemented in the form of a module, procedure, function, etc. that performs capabilities or operations described above. Software code can be stored in memory and executed by the processor. The memory is located inside or outside the processor, and may exchange data with the processor by various known means.

Meanwhile, the embodiments of the present disclosure may be implemented as a computer-readable code on a computer-readable recording medium. The computer-readable recording medium includes any type of recording device that stores data that may be read by a computer system. Examples of the computer-readable recording medium include ROM, RAM, CD-ROM, magnetic tape, floppy disks, and optical data storage devices. Further, the computer-readable recording medium may be distributed across network-connected computer systems, allowing the computer-readable code to be stored and executed in a distributed manner. Furthermore, functional programs, codes and code segments for implementing the embodiments can be easily inferred by programmers in the technical field to which the present disclosure pertains.

As described above, various embodiments of the present disclosure have been described. It is apparent to those skilled in the art that the various embodiments may be implemented in modified forms without departing from the essential characteristics of the present disclosure. Therefore, the disclosed embodiments should be considered in an illustrative rather than a restrictive sense. The scope of the present disclosure is defined by the claims rather than the foregoing description, and all differences coming within an equivalent scope thereof should be construed as being included herein.

Most of the terms used in the present disclosure are selected from commonly used terms in the relevant field. However, some terms are optionally chosen by the applicant and their meanings are described in detail in the following description as necessary. Therefore, the present disclosure should be understood based on the intended meaning of the terms rather than their simple names or literal meanings.

It will be apparent to those skilled in the art that the present disclosure may be embodied in other specific forms without departing from the essential characteristics of the present disclosure. Accordingly, the detailed description should not be construed as restrictive in all respects but should be considered illustrative. The scope of the present disclosure should be determined by a reasonable interpretation of the appended claims, and all changes coming within the equivalent scope of the present disclosure are intended to be included within the scope of the present disclosure.

What is claimed is:

1. A method for fusing heterogeneous sensors composed of a plurality of depth sensors and a three-dimensional localizer, the method comprising:
setting a separation position and angle of the plurality of depth sensors and the three-dimensional localizer;
performing a one-time calibration to estimate a relationship between coordinate systems of the plurality of depth sensors and the three-dimensional localizer, which are fixed to a frame according to the set separation position and angle;
integrating data of the plurality of depth sensors and the three-dimensional localizer into a single coordinate system using the one-time calibration result; and
simultaneously measuring real-time surface shape deformation of a target object and tracking positions and orientations of tools, using the integrated single coordinate system.

2. The method of claim 1, wherein the setting the separation position and angle of the plurality of depth sensors and the three-dimensional localizer sets a separation position and angle of the plurality of depth sensors, in consideration of a convergence angle between the plurality of depth sensors according to a separation distance between the plurality of depth sensors and a target object sensed by the plurality of depth sensors.

3. The method of claim 1, wherein the performing the one-time calibration comprises:
(a) measuring the surface points of a calibration plate, to which three or more retroreflective markers are attached and fixed at any position, based on each depth sensor coordinate system, using the plurality of depth sensors;
(b) calculating a normal vector of the calibration plate based on each depth sensor coordinate system using the surface points measured by each depth sensor;
(c) measuring three-dimensional positions of the three or more retroreflective markers based on the three-dimensional localizer coordinate system using the three-dimensional localizer;
(d) calculating a normal vector of the calibration plate based on the three-dimensional localizer coordinate system using the three-dimensional positions of the retroreflective markers measured by the three-dimensional localizer;
(e) calculating a plurality of three-dimensional rotation matrices using a plurality of normal vector sets obtained by repeatedly performing steps (a) to (d) two or more times;
(f) calculating a plurality of three-dimensional translation vectors using i) the plurality of normal vector sets, ii) the plurality of three-dimensional rotation matrices, iii) the surface points of the calibration plate, and iv) three-dimensional positions of the retroreflective markers; and
(g) generating a plurality of rigid body transformation matrices from the three-dimensional localizer coordinate system to the coordinate system of each of the plurality of depth sensors using the three-dimensional rotation matrix and the three-dimensional translation vector obtained through the calculation.

4. The method of claim 1, wherein the integrating the data of the plurality of depth sensors and the three-dimensional localizer into the single coordinate system comprises:
measuring surface shape data based on each depth sensor coordinate system from the plurality of depth sensors; and
transforming the surface shape data based on the single coordinate system set as the coordinate system of the three-dimensional localizer using a rigid body transformation matrix obtained in the one-time calibration process.

5. The method of claim 1, wherein simultaneously measuring the real-time surface shape deformation of the target object and the tracking the positions and orientations of the tools comprises:
obtaining surface shape measurement data transformed based on the single coordinate system; and
obtaining data on the positions and orientations of the tools represented based on the single coordinate system,
wherein the obtaining the surface shape measurement data and the obtaining the data on the positions and orientations of the tools may be simultaneously and repeatedly performed using parallel operation.

6. The method of claim 1, further comprising:
visualizing data measuring the real-time surface shape deformation of the target object and data simultaneously tracking the positions and orientations of the tools.

7. The method of claim 6, wherein the visualizing the data comprises:
rendering a three-dimensional model of the tool in a virtual space through three-dimensional positions of a retroreflective marker attached to the tool, rendering a three-dimensional model of the target object in a virtual space through three-dimensional surface shape information; and
updating the position and orientation of each three-dimensional model in real time.

8. The method of claim 6, wherein the visualizing the data further comprises:
checking whether a retroreflective marker attached to the tool is visible, thereby visually expressing the dislodgement of the tool when the tool is out of the field of view of a fusion apparatus of the heterogeneous sensors.

9. The method of claim 1, wherein the plurality of depth sensors and the three-dimensional localizer are configured in an orthogonally polarized state based on the similarity between (i) a wavelength range of light used by the plurality of depth sensors and (ii) a wavelength range of light used by the three-dimensional localizer.

10. An apparatus for fusing heterogeneous sensors, the apparatus comprising:
a plurality of depth sensors;
a three-dimensional localizer; and
a processor,
wherein the plurality of depth sensors and the three-dimensional localizer are fixed to a frame according to a set separation position and angle,
wherein the processor performs a one-time calibration to estimate a relationship between coordinate systems of the plurality of depth sensors and the three-dimensional localizer, which are fixed to the frame, integrates data of the plurality of depth sensors and the three-dimensional localizer into a single coordinate system using the one-time calibration result,
simultaneously measures real-time surface shape deformation of a target object, and tracks positions and orientations of tools.

11. The apparatus of claim 10, wherein the separation position and angle are set, in consideration of a convergence angle between the plurality of depth sensors according to a separation distance between the plurality of depth sensors and a target object sensed by the plurality of depth sensors.

12. The apparatus of claim 10, wherein the processor
measures the surface points of a calibration plate, to which three or more retroreflective markers are attached and fixed at any position, based on each depth sensor coordinate system, using the plurality of depth sensors,
calculates a normal vector of the calibration plate based on each depth sensor coordinate system using the surface points measured by each depth sensor,
measures three-dimensional positions of the three or more retroreflective markers based on the three-dimensional localizer coordinate system using the three-dimensional localizer,
calculates a normal vector of the calibration plate based on the three-dimensional localizer coordinate system using the three-dimensional positions of the retroreflective markers measured by the three-dimensional localizer,
calculates a plurality of three-dimensional rotation matrices using a plurality of normal vector sets obtained by repeating the process of calculating the normal vector,
calculates a plurality of three-dimensional translation vectors using i) the plurality of normal vector sets, ii) the plurality of three-dimensional rotation matrices, iii) the surface points of the calibration plate, and iv) three-dimensional positions of the retroreflective markers, and
generates a plurality of rigid body transformation matrices from the three-dimensional localizer coordinate system to the coordinate system of each of the plurality of depth sensors using the three-dimensional rotation matrix and the three-dimensional translation vector obtained through the calculation.

13. The apparatus of claim 10, wherein the processor measures surface shape data based on each depth sensor coordinate system from the plurality of depth sensors, and transforms the surface shape data based on the single coordinate system set as the coordinate system of the three-dimensional localizer using a rigid body transformation matrix obtained in the one-time calibration process.

14. The apparatus of claim 10, wherein the processor obtains surface shape measurement data transformed based on the single coordinate system, and obtains data on the positions and orientations of the tools represented based on the single coordinate system, thereby measuring surface shape deformation in real time and tracking the positions and orientations of the tools.

15. The apparatus of claim 14, wherein the processor simultaneously and repeatedly performs, by the heterogeneous sensor fusion apparatus, a process of obtaining the surface shape measurement data and a process of obtaining the data on the positions and orientations of the tools through parallel operation.

16. The apparatus of claim 10, wherein the processor visualizes data
simultaneously measuring the real-time surface shape deformation of the target object and tracking the positions and orientations of the tools.

17. The apparatus of claim 16, wherein the processor renders a three-dimensional model of the tool in a virtual space through three-dimensional positions of a retroreflective marker attached to the tool, rendering a three-dimensional model of the target object in a virtual space through three-dimensional surface shape information, and updating the position and orientation of each three-dimensional model in real time.

18. The apparatus of claim 16, wherein the processor checks whether a retroreflective marker attached to the tool is visible, thereby visually expressing the dislodgement of the tool when the tool is out of the field of view of the fusion apparatus of the heterogeneous sensors.

19. The apparatus of claim 10, wherein the plurality of depth sensors and the three-dimensional localizer are configured in an orthogonally polarized state based on the similarity between (i) a wavelength range of light used by the plurality of depth sensors and (ii) a wavelength range of light used by the three-dimensional localizer.

20. The apparatus of claim 19, wherein the orthogonally polarized state is based on polarizing films or polarizing lenses attached to an illumination part or a camera unit of each of the plurality of depth sensors and the three-dimensional localizer.
